(19) United States
(12) Plant Patent Application Publication
(10) Pub. No.: US 2017/0118896 P1
(43) Pub. Date: Apr. 27, 2017

(54) Mandevilla plant named 'OOSMARE1'

(50) ;

(71) Applicant: Houdstermaatschappij Oosterom B.V., Waddinxveen (NL)

(72) Inventor: Petrus Cornelis Vijverberg, Naaldwijk (NL)

(73) Assignee: Houdstermaatschappij Oosterom B.V., Waddinxveen (NL)

(21) Appl. No.: 15/330,656

(22) Filed: Oct. 24, 2016

(30) Foreign Application Priority Data
Oct. 26, 2015 (QZ) 2015/2459

(51) Int. Cl. A01H 5/00

(52) U.S. Cl. PLT/232

(57) ABSTRACT

A new and distinct Mandevilla cultivar named ‘OOSMARE1’ which is characterized by a compact growth habit, dark green and glossy foliage, large bright red flowers, a continuous bloom habit, as well as the stability of these characteristics from generation to generation.

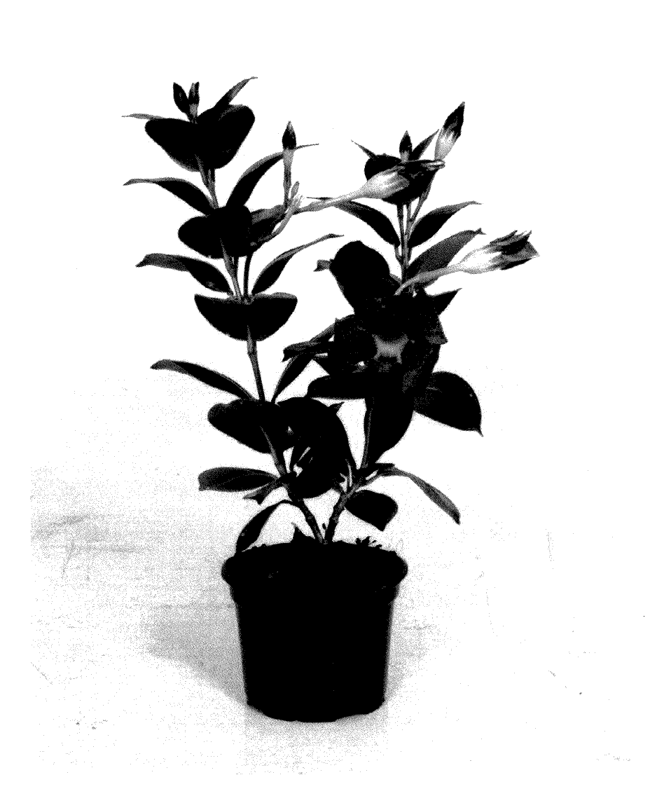

### CROSS REFERENCE TO RELATED APPLICATIONS

This application claims priority to the Community Plant Variety Rights application number 2015/2459, filed Oct. 26, 2015, which is herein incorporated by reference.

Latin name of the genus and species: The Latin name of the genus and species of the novel variety disclosed herein is Mandevilla sanderi (Hemsl.) Woodson.

Variety denomination: The inventive variety of Mandevilla disclosed herein has been given the variety denomination ‘OOSMARE1’.

### BACKGROUND OF THE INVENTION

Parentage: Over the course of many years, the inventor sourced various Mandevilla sanderi (all non-patented) plants with flower colors ranging from white to pink to red. From this plant material, the inventor made selections of individual plants as well as single branches of individual plants, based on flower color, earliness of flowering, compactness of the plant, persistence of flowering, persistence in keeping flower color during the flowering. Many individual varieties were identified, and some were isolated for further evaluation. The candidate variety is the result of this selection process, and was first observed in the spring of 2014 in Westland, The Netherlands.

Asexual Reproduction: Asexual reproduction of the new cultivar ‘OOSMARE1’, by way of softwood stem cuttings, was first initiated in the summer of 2014 in Westland, The Netherlands. Through more than 10 subsequent generations, the unique features of this cultivar have proven to be stable and true to type.

### SUMMARY OF THE INVENTION

The cultivar ‘OOSMARE1’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘OOSMARE1’. These characteristics in combination distinguish ‘OOSMARE1’ as a new and distinct Mandevilla cultivar:
1. Mandevilla ‘OOSMARE1’ exhibits a compact growth habit; and
2. Mandevilla ‘OOSMARE1’ exhibits dark green and glossy foliage; and
3. Mandevilla ‘OOSMARE1’ exhibits large bright red flowers; and
4. Mandevilla ‘OOSMARE1’ exhibits continuous flowering.

### BRIEF DESCRIPTION OF THE FIGURES

The FIGURE illustrates, as nearly true as it is reasonably possible to make the same in color photographs of this type, an exemplary plant of ‘OOSMARE1’ in flower, grown in a commercial greenhouse in Honselersdijk, the Netherlands.

### BOTANICAL DESCRIPTION OF THE PLANT

The following observations and measurements were made in October of 2016 and describe a mature ‘OOSMARE1’ plant grown in a greenhouse in Westland, The Netherlands. Plants were watered with overhead irrigation and fertilizer was applied using fertigation. No pest or disease measures were taken.

Those skilled in the art will appreciate that certain characteristics will vary with older or, conversely, with younger plants. ‘OOSMARE1’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climactic and cultural conditions. Color notations are based on The Royal Horticultural Society Colour Chart, The Royal Horticultural Society, London, 2015 (sixth edition).

A botanical description of ‘OOSMARE1’ and comparisons with other varieties of Mandevilla are provided below.
Plant description:
   Growth habit.—Compact vining perennial; upright and spreading.
   Average height.—
   Plant spread.—
   Growth rate.—Moderate.
   Plant vigor.—Very vigorous.
   Propagation method.—Softwood stem cuttings; rooting is in initiated in approximately 10 to 14 days under greenhouse conditions.
   Time to produce a finished plant.—Approximately 6 to 9 months, depending on pot size, is required to produce a fully rooted and marketable plant when grown in a greenhouse with a minimum ambient temperature of 14 degrees Celsius.
   Disease and pest resistance or susceptibility.—Plants have not been observed to be susceptible or resistant to pathogens and pests common to Mandevilla sanderi.
   Environmental tolerances.—Adapt to at least USDA Zones 10 and 12; high tolerance to rain; moderate tolerance to wind; low tolerance to drought.
Root system:
   General.—Freely branching; moderately dense rooting.
   Texture.—Fibrous. 6p2 Color.—Light brown.
Stem:
   Branching habit.—Basally branching main stem; no lateral branches present.
   Number of main stems per plant.—Two.
   Number of lateral branches per plant.—None.
   Appearance; cross-section.—Rounded.
   Internode length.—
   Aspect.—Upright; angle is approximately 75 to 80 degrees from horizontal plane.
   Strength.—Medium.
   Color, juvenile.—Yellow-green.
   Color, mature.—Greyed-orange.
   Texture.—Glabrescent.
Foliage:
   Arrangement.—Opposite.
   Quantity.—Approximately 24 leaves per stem, as observed.
   Lamina.—Shape — Broad elliptical. Aspect — Nearly flat; slightly reflexed. Apex — Broad acuminate. Base — Broad cuneate to obtuse. Margin — Entire. Pubescence, texture and luster of adaxial surface — Glabrous, smooth, and very glossy. Pubescence, texture and luster of abaxial surface — Pubescent, smooth, and matte. Color — Juvenile foliage, adaxial surface — Yellow-green and suffused with anthocyanin intonations. Juvenile foliage, abaxial surface — Light yellow-green and suffused with anthocyanin intonations. Mature foliage, adaxial surface — Dark green to dark yellow-green. Mature foliage, abaxial surface — Light yellow-green. Venation — Pattern — Pinnate. Venation color, adaxial surface — Dark green to dark yellow-green; same color as adaxial leaf surface. Venation color, abaxial surface — Dark yellow-green; darker than the adaxial leaf surface.
   Petiole.—Color — Light yellow-green. Texture — Glabrescent.
Inflorescence:
   Inflorescence arrangement.—Terminal and axillary racemes bearing 1 to 4 flowers.
   Natural flowering season.—Summer through autumn in USDA Hardiness Zones 10 through 12.
   Time to flower.—Approximately 2 months from planting a rooted cutting.
   Peduncle.—Aspect — Upward and outward. Strength — Medium. Texture and luster — Glabrous and very slightly glossy. Color — Yellow-green.
Bud:
   Shape.—Oblong, elongated; long pointed ovoid at the distal end.
   Pubescence, texture and luster.—Glabrous, smooth and moderately glossy.
   Color.—Yellow-green at the proximal end and fading to red at the distal end.
Flower:
   Flowering habit.—Freely flowering; recurrent.
   Shape.—Salverform.
   Aspect.—Flowers are upward to outward facing.
   Fragrance.—No fragrance.
   Lastingness.—3 to 5 days.
   Persistent.—Persistent.
   Pedicels.—Aspect — Upward and outward. Strength — Medium. Texture and luster — Glabrous and very slightly glossy. Color — Yellow-green.
   Petals.—Quantity of Petals — 5. Arrangement — Single whorl; petals imbricate and fused into an elongated tube at the base. Shape of petal lobes — Obovate to near orbicular. Apex — Bluntly acute. Base — Truncate; fused at the base. Margin — Entire; moderately undulated. Aspect — Heavily reflexed. Pubescence, texture and luster of upper surface — Glabrous, velvety and matte. Pubescence, texture and luster of lower surface — Glabrous, velvety and matte. Petal lobe color when opening, upper surface — Red. Petal lobe color when opening, lower surface — Red. Petal lobe color when fully opened, upper surface — Red. Petal lobe color when fully opened, lower surface — Red. Inner corolla tube color, when fully opened — Yellow.
   Sepals.—Arrangement — Sepals fused at the base forming a tube; free portion of the sepals arranged in a whorl. Quantity — 5. Apex — Acute. Base — Truncate; fused at the base. Margin — Entire; not undulated. Pubescence, texture and luster, upper surface — Smooth; glabrous. Pubescence, texture and luster, lower surface — Smooth; glabrous. Color when opening, upper surface — Yellow-green suffused with red. Color when opening, lower surface — Yellow-green suffused with red. Color when fully opened, upper surface — Yellow-green suffused with red. Color when fully opened, lower surface — Yellow-green suffused with red.
Reproductive organs:
   Androecium.—Five stamens adnate to the base of the corolla tube with linear anthers; anthers basifixed. Pollen not observed.

2 Gynoecium.—One pistil with a conical stigma; stigma and style are yellow-green in color; ovary is inferior with an elongated shape and colored yellow-green.
Seed and fruit: None observed.
Comparisons with the parent plants: Plants of the new cultivar ‘OOSMARE1’ differ from the unnamed Mandevilla sanderi (Hemsl.) Woodson parent by the characteristics described in Chart 1.

CHART 1

| Characteristic | ‘OOSMARE1’ | Unnamed seed parent |
|---|---|---|
| Time to flower. | Flowers 2 to 3 weeks | Flowers 2 to 3 weeks |
| | earlier than the species. | later than ‘OOSMARE1’. |
| Growth habit. | More compact than the | Less compact than |
| | species. | ‘OOSMARE1’. |
| Flower color, when | More bright red. | Less bright red. |
| opening. | | |
| Flower color fading. | Not fading. | Fades to a light pink, |
| | | almost white. |
| Lastingness of | Longer than the | Shorter than |
| flowers. | species. | ‘OOSMARE1’. |

Comparison with the most similar Mandevilla cultivar known to the inventor: Comparison data not available at the time of filing the instant application.

That which is claimed is:

1. A new and distinct variety of Mandevilla plant named ‘OOSMARE1’, substantially as described and illustrated herein.
